(19) United States
(12) Patent Application Publication
(10) Pub. No.: US 2012/0239076 A1
(43) Pub. Date: Sep. 20, 2012

(54) RECTAL STIMULATION DEVICE

(76) Inventor: George J. Cisko, JR., Spring Grove, IL (US)

(21) Appl. No.: 13/510,469

(22) PCT Filed: Nov. 19, 2010
(86) PCT No.: PCT/US10/57444

§ 371 Date: Jun. 1, 2012

Related U.S. Application Data
(60) Provisional application No. 61/253,101, filed on Oct. 20, 2009.

(51) Int. Cl. A61M 29/02

(52) U.S. Cl. 606/197

(57) ABSTRACT

A rectal stimulation device for stimulating peristalsis includes a balloon having an interior, a squeeze bulb having an interior and an insertion portion having a distal end and providing flow communication between the interior of the squeeze bulb and the interior of the balloon. The balloon, the squeeze bulb and the insertion portion define a closed fluid system. The balloon overlies the insertion portion distal end. A fluid is disposed in the closed fluid system. The distal end and the balloon are inserted into a rectum of a user, and pressure is applied to the squeeze bulb to urge fluid from the interior off the squeeze bulb to the interior of the balloon to distend the balloon.

### BACKGROUND

The present disclosure is directed to a device to initiate peristalsis using direct rectal stimulation.

Individuals with neurogenic bowel dysfunction, such as that caused by spinal cord injury, small bowel disease, multiple sclerosis, and the like, typically require a bowel regimen or routine to initiate or facilitate peristalsis (organized muscle contraction) of the bowel. Depending on the level of injury, the individual may require direct stimulation of the nerves of the rectum to initiate peristalsis and subsequent defecation.

Self-administered manual stimulation of the rectum is difficult, especially for individuals with limited dexterity. Additionally, if the individual requires assistance from a caregiver with the bowel regimen, dignity may be compromised for both the individual and the caregiver.

Mechanical devices used to initiate peristalsis are generally made of hard metal and/or plastic and can be cold and uncomfortable to use. Furthermore, curvature of the rectal cavity sometimes prevents adequate stimulation of the nerves of the rectum when these hard devices are used. In addition, the degree of such stimulation is not easily modified or changed with changing posture or bowel habits.

Accordingly, there is a need for a rectal stimulation device which increases an individual's comfort and dignity during the bowel regimen while providing a modifiable source of rectal stimulation in order to initiate peristalsis.

### BRIEF SUMMARY

A rectal stimulation device for initiating peristalsis of the bowel includes a balloon, a squeeze bulb and a hollow insertion portion. The insertion portion has a distal end, and the balloon overlies the distal end the balloon. The squeeze bulb, the insertion portion and the balloon define a closed fluid system.

A fluid is disposed in the closed fluid system. The insertion portion distal end and the balloon are inserted into a rectum of a user, and pressure is applied to the squeeze bulb to urge fluid from the squeeze bulb to the balloon to distend the balloon. Balloon distension within the rectum initiates peristalsis within the user.

These and other features and advantages of the present device will be apparent from the following detailed description, in conjunction with the appended claims.

### BRIEF DESCRIPTION OF THE SEVERAL VIEWS OF THE DRAWINGS

The benefits and advantages of the present device will become more readily apparent to those of ordinary skill in the relevant art after reviewing the following detailed description and accompanying drawings, wherein:

FIG. 1 is a sagittal section of a human female;

FIGS. 2A and 2B are top and side (plane and elevational) views of an embodiment of the rectal stimulation device;

FIG. 3 is a side view of another embodiment of the rectal stimulation device; and

FIGS. 4A and 4B illustrate the rectal stimulation device in use, with the balloon in an undistended state (FIG. 4A) and a distended state (FIG. 4B).

### DETAILED DESCRIPTION

While the present device is susceptible of embodiment in various forms, there is shown in the drawings and will hereinafter be described a presently preferred embodiment with the understanding that the present disclosure is to be considered an exemplification of the device and is not intended to limit the disclosure to the specific embodiment illustrated.

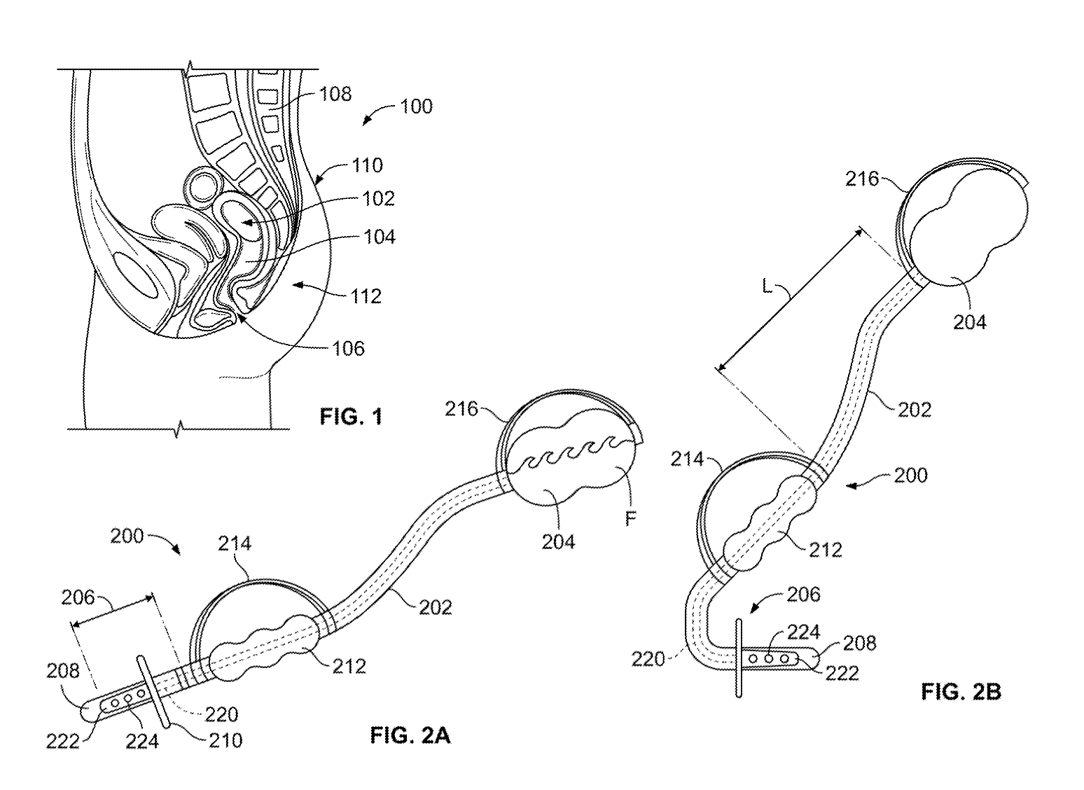

Referring now to the figures and in particular to FIG. 1, there is illustrated the sagittal section of a human female torso 100. The large bowel 102 is located superior to the rectum 104 and the anus 106. The curvature of the spine 108 and pelvic region, generally at 110, as well as other anatomical organs, cause the rectum to be curved. The present rectal stimulation device is configured to stimulate the rectum by providing gentle, modifiable, and flexible stimulation through at least a portion of the curvature of the rectal region 112.

Turning now to FIGS. 2A and 2B, there is shown an embodiment of the present rectal stimulation device 200 for stimulating peristalsis. The device 200 includes a squeeze bulb 204, a fluid conduit 202, an insertion portion 206 and a balloon 208. The illustrated, exemplary insertion portion 206 is hollow. The rectal stimulation device 200 is a closed system and contains a biocompatible fluid or gel F, preferably incompressible, such as saline, water, glycerin, mineral oil or the like.

The squeeze bulb 204 is a rubber or elastic inflation bulb in fluid communication with the insertion portion 206 through the fluid conduit 202.

The insertion portion 206 includes a proximal end 220 and a distal end 222, and can include a stop 210 intermediate the proximal and distal ends 220, 222 that is configured to prevent over-insertion of the rectal stimulation device 200 into the rectum. The insertion portion 206 is sufficiently rigid to facilitate insertion into the rectum, but can have some measure of flexibility or malleability to allow bending thereof to facilitate bending or shaping the device for comfort as it is inserted into the anus/rectum.

The distal end 222 has a relatively small diameter of about ⅜ inch to about ⅝ inch and preferably about ½ inch to facilitate insertion into the anal opening 106. One or more openings or holes 224 are formed in the portion 206 at the distal end 222. It will be appreciated by those skilled in the art that other configurations of the insertion portion 206 can be used to provide a flow path for the fluid from the conduit to the interior of the balloon. For example, a solid, channeled element with openings to provide a path for fluid communication from the interior of the tube/bulb to the exterior of the portion (and the interior of the overlying balloon) are contemplated, as are other hollow and solid flow path providing configurations.

The balloon 208 is a soft, resilient, biocompatible material that resists tearing, decomposition, and/or seepage of the fluid or gel F. The balloon 208 is formed integral with or covers at least the distal end 222 of the insertion portion 206. In this manner, the balloon 208 overlies the openings 224 in the distal end 222.

The fluid conduit 202 can be provided as a malleable tube that extends between the squeeze bulb 204 and the insertion portion 206. The conduit 202 can be bendable/malleable and able to hold its shape once bent. This permits bending the device 200 into a desired shape to facilitate comfort and ease of use. The conduit 202 can have a variety of lengths L to, for example, accommodate individuals whether ambulatory or not, for self-administration. Accordingly, it is anticipated that fluid conduits 202 will have lengths between about 12 inches and 24 inches.

The device 200 can include a hand grip 212 positioned along the conduit 202, proximal to the insertion portion. A flexible hand strap 214 can be provided at the grip 212 to permit manipulating or maneuvering and holding the device 200. The strap 214 can be, for example, a mechanical hook and loop fastening system, an elastic strap or the like. An additional strap 216 can be provided at the squeeze bulb 204.

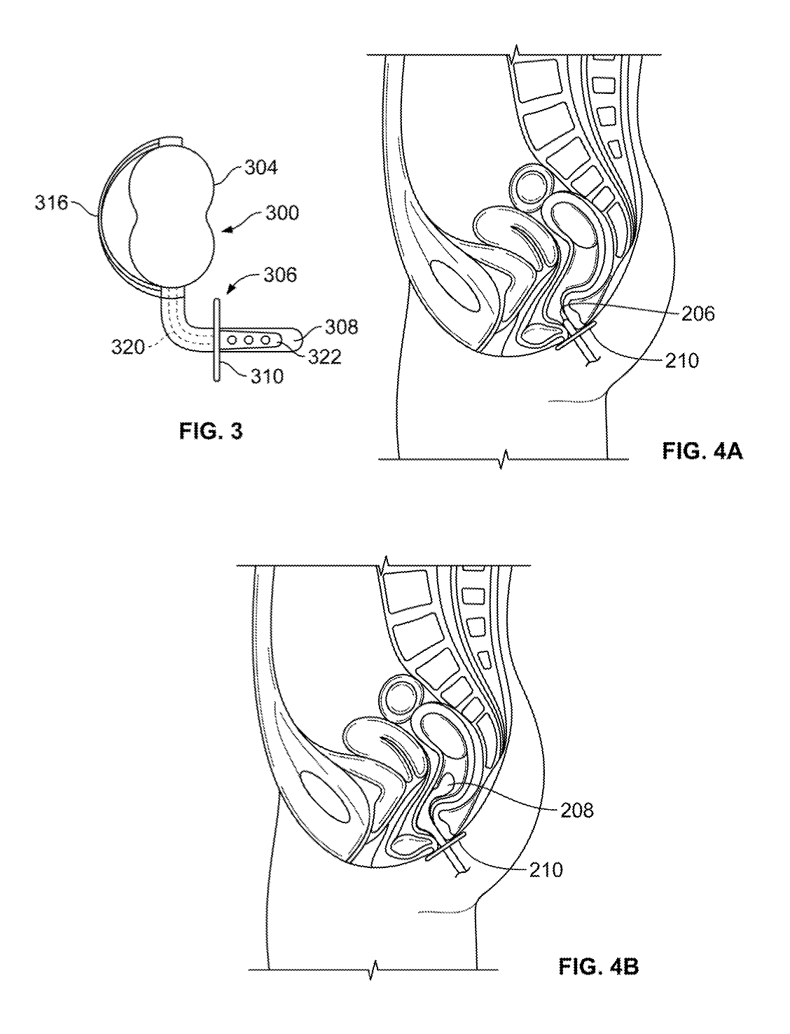

Referring to FIGS. 4A and 4B, in use, the insertion portion 206 is inserted into the rectum with the balloon 208 in the un-distended condition (FIG. 4A). The stop 210 prevents over insertion of the portion 206. A lubricant can be used in conjunction with the balloon 208 and insertion portion 206 to facilitate insertion of the device 200 into the rectum.

Once the device 200 is in place, the user squeezes the squeeze bulb 204, which forces the fluid F from the bulb 204, through the conduit 202, through the openings 224 in the distal end 222, and into the balloon 208. The balloon 208 fills with the fluid F and distends (FIG. 4B). Upon release of the squeeze bulb 204, the balloon 208 returns to its un-distended shape. Distention of the balloon 208 causes stimulation of the rectum. Distention occurs with, and is proportional to, each compression and release of the squeeze bulb 204. The balloon 208 can be configured or shaped to distend more in one direction length than the other, e.g., configured to distend more in width or length than the other. It will be understood that distention occurs in relation to the size and shape of a rectum in which the balloon 208 is distended.

As seen in FIG. 3, in an alternate embodiment, the device 300 has a shortened conduit or essentially no conduit, such that the squeeze bulb 304 is effectively mounted to the proximal end 320 of the insertion portion 306. It will be appreciated that in such an embodiment 300 a hand grip may not be provided and manipulation of the device 300 may be carried out using the bulb 304, insertion portion 306 and a strap 316.

The insertion portions 206, 306 can be formed having various shapes to facilitate how and by whom the device is used. For example, the insertion portion 206 can have a J-shape as seen in FIG. 2B, or it can have more of an L-shape, as seen in FIG. 3. It will be understood that all such shapes are within the scope and spirit of the present disclosure.

It will also be appreciated that the illustrated devices 200, 300 are closed and sealed devices. That is, in use, the system is closed to the introduction or removal of fluid. They are also sealed in that there are no valves, openings, fill ports or the like, by which fluid can be introduced into or removed from the device. It is anticipated that an embodiment of the device can be a non-sealed system configured with valves, openings, fill ports or the like to facilitate such introduction and/or removal of fluid. However, even such a non-sealed system would be closed in use.

In addition, an embodiment of the device can be configured so the that insertion portion is removable/replaceable. It is anticipated that in such a system, a valve is positioned between the insertion portion and the squeeze bulb, likely proximal to the insertion portion to permit isolating the insertion portion for removal. It is also contemplated that the balloon can be replaceable. It is further anticipated that the entirety of the insertion portion can be removable/replaceable. Further, the device can be configured with an expandable sleeve fitted onto (over) the balloon so that the balloon can be reused and the sleeve disposed of after each use to maintain cleanliness of the balloon and device.

Those skilled in the art will appreciate the advantages provided by the present rectal stimulation device. The device allows individuals with limited dexterity to perform a bowel regimen easily and comfortably. It also allows caregivers who assist in such bowel regimens flexibility and maneuverability while helping the individual maintain dignity.

All patents referred to herein, are incorporated herein, in their entirety, by reference, whether or not specifically done so within the text of this disclosure.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present device. It is to be understood that no limitation with respect to the specific embodiments illustrated is intended or should be inferred. The disclosure is intended to cover by the appended claims all such modifications as fall within the scope of the claims.

1. A rectal stimulation device for stimulating peristalsis comprising:
a balloon having an interior;
a squeeze bulb having an interior; and
an insertion portion having a distal end, the balloon, the squeeze bulb and the insertion portion defining a closed fluid system, the balloon overlying the insertion portion distal end, the insertion portion providing flow communication between the interior of the squeeze bulb and the interior of the balloon; and
a fluid disposed in the closed fluid system,
wherein the distal end and the balloon are inserted into a rectum of a user, and pressure is applied to the squeeze bulb to urge fluid from the interior of the squeeze bulb to the interior of the balloon to distend the balloon.

2. The rectal stimulation device of claim 1 wherein the fluid is a biocompatible fluid or a gel.

3. The rectal stimulation device of claim 2 wherein the fluid is water, saline solution, glycerin, mineral oil or the like.

4. The rectal stimulation device of claim 1 including a stop to prevent over-insertion of the insertion portion.

5. The rectal stimulation device of claim 1 including a fluid conduit extending between the squeeze bulb and the insertion portion.

6. The rectal stimulation device of claim 1 wherein the balloon is configured to distend more in either an elongated direction or a width direction.

7. The rectal stimulation device of claim 5 wherein the fluid conduit is bendable and is formed so as to hold its shape when bent.

8. The rectal stimulation device of claim 1 wherein the insertion portion is formed having a straight or curved shape.

9. The rectal stimulation device of claim 1 including a hand grip.

10. The rectal stimulation device of claim 9 wherein the hand grip is disposed between the squeeze bulb and the insertion portion.
